(19) United States
(12) Patent Application Publication
(10) Pub. No.: US 2015/0306281 A1
(43) Pub. Date: Oct. 29, 2015

(54) DEVICES AND METHODS FOR INHIBITING STENOSIS, OBSTRUCTION, OR CALCIFICATION OF A NATIVE HEART VALVE, STENTED HEART VALVE OR BIOPROSTHESIS

(71) Applicant: Nalini Marie Rajamannan, Chicago, IL (US)

(72) Inventor: Nalini Marie Rajamannan, Chicago, IL (US)

(21) Appl. No.: 14/687,479

(22) Filed: Apr. 15, 2015

Related U.S. Application Data
(63) Continuation-in-part of application No. 14/263,438, filed on Apr. 28, 2014.

(51) Int. Cl. A61L 27/54; A61L 27/36; A61F 2/24

(52) U.S. Cl. CPC A61L 27/54; A61F 2/2412; A61F 2/2418; A61L 27/3625; A61F 2250/0067; A61L 2400/02; A61L 2430/20; A61L 2300/416

(57) ABSTRACT

The present invention relates to methods for inhibiting stenosis, obstruction, or calcification of a valve following implantation of a valve prosthesis or a native valve which develops disease via the Lrp5/Wnt Pathway in the presence of elevated lipids due to elevated Low Density Lipoprotein. This invention involves dispensing a combination of medications to target inflammation and attachment of the target cell and the secondary drugs to inhibit proliferation and calcification on an elastical stent, gortex graft or valve leaflet. The combination therapy inhibits bioprosthesis and native valve calcification with improvement of the longevity of the prosthetic material including the stent, the native valve, and the gortex covering. The valve prosthesis and or gortex graft is mounted on the elastical stent or prosthesis such that the elastical stent is connected to the valve.

### RELATED APPLICATIONS

This application is a continuation-in-part of U.S. application Ser. No. 14/263,438, filed on Apr. 28, 2014, which is pending, the entirety of which is hereby incorporated by reference.

### FIELD OF THE INVENTION

The invention relates to devices and methods for inhibiting stenosis, obstruction, or calcification of native heart valves and heart valve bioprostheses.

### BACKGROUND OF THE INVENTION

The heart is a hollow, muscular organ that circulates blood throughout an organism's body by contracting rhythmically. In mammals, the heart has four-chambers situated such that the right atrium and ventricle are completely separated from the left atrium and ventricle. Normally, blood flows from systemic veins to the right atrium, and then to the right ventricle from which it is driven to the lungs via the pulmonary artery. Upon return from the lungs, the blood enters the left atrium, and then flows to the left ventricle from which it is driven into the systematic arteries.

Four main heart valves prevent the backflow of blood during the rhythmic contractions: the tricuspid, pulmonary, mitral, and aortic valves. The tricuspid valve separates the right atrium and right ventricle, the pulmonary valve separates the right atrium and pulmonary artery, the mitral valve separates the left atrium and left ventricle, and the aortic valve separates the left ventricle and aorta. Generally, patients having an abnormality of a heart valve are characterized as having valvular heart disease.

A heart valve can malfunction either by failing to open properly (stenosis) or by leaking (regurgitation). For example, a patient with a malfunctioning aortic valve can be diagnosed with either aortic valve stenosis or aortic valve regurgitation. In either case, valve replacement by surgical means may be a possible treatment. Replacement valves can be autografts, allografts, or xenografts as well as mechanical valves or valves made partly from valves of other animals, such as pig or cow. Unfortunately, over time, the replacement valves themselves are susceptible to problems such as degeneration, thrombosis, calcification, and/or obstruction. Furthermore, the process of valve replacement may cause perforation in the surrounding tissue, leading also to stenosis, degeneration, thrombosis, calcification, and/or obstruction.

Thus, new methods and prostheses for inhibiting stenosis, obstruction, or calcification of heart valves are needed.

### SUMMARY OF THE INVENTION

The foregoing problems are addressed by the method for inhibiting stenosis, obstruction, or calcification of a native valve and a valve prosthesis, both in accordance with the invention.

In a first aspect of the invention the method slows the progression of bicuspid aortic valve (BAV) calcification, tricuspid aortic valve calcification (TAV), transcutaneous aortic valve replacement (TAVR), surgical bioprosthetic aortic valve replacement (SBAVR), mitral valve myxomatous degeneration (MVMD) via the activation of the Wnt pathway via the cleavage of Notch1 protein and the phosphorylation of glycogen synthase kinase which in turn releases beta catenin to the nucleus to activate bone and cartilage formation the heart valve and or prosthesis.

In another aspect of the invention several therapeutic medical therapies that slow the progression of stenosis, obstruction, calcification and or regurgitation of the mitral valve are provided. Specifically, in the presence of hyperlipidemia, there is a decrease in Nitric oxide and Wnt3a is farnesylated in order for the secretion of Wnt, which in turns binds to Lrp5, in addition Notch1 is spliced and inactivated in order for the CBFA1 regulation of cell proliferation extracellular matrix protein synthesis to initiate bone formation by activation of osteogenic bone program.

In further aspects, the invention may be set out in the following numbered clauses:

1. A method for inhibiting stenosis, obstruction, or calcification of a bioprosthetic valve implanted in a patient comprising:
implanting a bioprosthetic valve in a patient to replace a natural heart valve;
following implantation administering an effective amount of an anti-hyperlidemic agent in combination with a PCSK9 antibody; and
causing the inhibition of stenosis, obstruction, or calcification of the bioprosthetic valve or natural valve or both.
   2. The method according to clause 1, wherein said effective amount of anti-hyperlidemic agents is selected from 10 mg to 80 mg of Atorvastatin, 10 mg to 40 mg of Simvastatin, 5 mg to 40 mg of Rosuvastatin, 20 mg to 80 mg of Pravastatin, 1 mg to 4 mg of Pitavastatin and combinations of the foregoing.
   3. The method of clause 3 wherein an initial dose of PCSK9 is from 0.25 mg/kg to about 0.5 mg/kg.
   4. The method of clause 3 wherein a subsequent dose of PCSK9 is from about 1 mg/kg to about 1.5 mg/kg.
   5. The method of clause 4 wherein said initial dose and subsequent dose are separated in time by about one week.
   6. The method of clause 1 further comprising administering an effective amount of a farnesyltransferase inhibitor.
   7. The method according to clause 6 wherein said farnesyltransferase inhibitor comprises lonafarnib and said effective amount comprises from 115 mg/m² to 150 mg/m².
   8. The method of clause 7 further comprising administering Zetia in an amount equal to of 10 mg.
   9. The method of clause 1, wherein the bioprosthetic valve is an aortic bioprosthetic valve.
   10. The method of clause 1, wherein the bioprosthetic valve is a bioprosthetic mitral valve.
   11. The method of clause 1, wherein the bioprosthetic valve is a bioprosthetic pulmonic valve.
   12. The method of clause 1, wherein the bioprosthetic valve is bioprosthetic tricuspid valve.
   13. The method of clause 1, wherein the bioprosthetic valve comprises one or more cusps of biological origin.
   14. The method of clause 13, wherein the one or more cusps is porcine, bovine, or human.
   15. The method of clause 13, further comprising introducing a nucleic acid encoating a nitric oxide synthase into the one or more cusps.
   16. The method according the clause 13, further comprising introducing a drug eluting treating encoating the one or more cusps on both sides with an anti-proliferative and anti-calcification treatment.
   17. The method of clause 1 further comprising administering aspirin in an amount equal to 80 mg/day.
   18. The method of clause 1 further comprising administering an effective amount of an oral P2Y12 inhibitor.
   19. The method of clause 18 wherein said P2Y12 inhibitor is selected from Clopidogrel, Prasugrel, Ticagrelor and combinations of the foregoing.
   20. The method of clause 19 wherein said effective amount of Clopidogrel is a loading dose of 300 mg at the time of implantation and a maintenance dose of 75 mg/day thereafter.
   21. The method of clause 19 wherein said effective amount of Prasugrel is a loading dose of 60 mg at the time of implantation and a maintenance dose of 10 mg/day thereafter.
   22. The method of clause 19 wherein said effective amount of Ticagrelor is a loading dose of 180 mg at the time of implantation and a maintenance dose of 90 mg two times per day thereafter.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 is an illustration that depicts the signaling mechanisms of valve calcification in the presence of hyperlidemia.

FIG. 2 are images showing preliminary data of native valve atherosclerosis in the presence of a cholesterol diet, lithium chloride diet, and the attenuation of the valve leaflet with the treatment of atorvastatin in a mouse valve leaflet that has no LDL receptors.

FIG. 3, Panel A depicts the in vitro data of the direct treatment of myofibroblast cells with the increasing dose of lithium chloride increasing cell proliferation.

FIG. 3, Panel B is the inhibition of DKK1 with atorvastatin and the direct inhibition of Lrp5.

FIG. 4, demonstrates the characterization of the eNOS phenotype as defined by histology, RTPCR and echocardiography.

FIG. 4, Panel A depicts the histology for BAV.

FIG. 4, Panel B depicts the semi-quantitative RTPCR from the BAV eNOS^{-/-} mice, and echocardiographic data for the bicuspid vs. tricuspid aortic valves.

FIG. 4, Panel C is a table depicting the echocardiography from the eNOS null mouse on different diets.

FIG. 5 is a schematic view showing the cell layers which develop the disease process in the native valve leaflet via the signaling between the endothelial cell to the myofibroblast cell in the presence of hyperlipidemia to activate the secretion of Wnt to turn on the Lrpb5 receptor which in turn activates bone formation in the native myofibroblast cell and the different inhibitors and oral agents to slow the progression of disease.

FIG. 6 is a schematic view showing an aorta having the aortic valve with the cells therein the native valve or the bioprosthesis, in which the aorta surrounding the stent has been partially blocked by stenosis secondary to vascular smooth muscle cell proliferation and differentiation to bone forming cells after injury from the stent adjacent to the aorta, and c-kit stem cell or the in vivo myofibroblast cell proliferation and differentiation to bone formation cells secondary to inflammation and homing of c-kit stem cells to become bone forming cells and the effect of medications including statins, proprotein convertase subtilisin kexin type 9 antagonist antibody (“PCSK9 antibody”), and a farnesyltransferase (“FTI”) inhibitor.

FIG. 7 depicts pannus formation and calcification in the explanted valves from human patients at the time of surgical valve replacement of a failed bioprosthetic heart valve secondary to proliferating mesenchymal stem cells attaching to the valve and stent which calcifies and causes valve leaflet and stent destruction.

FIG. 8 is a graph, which demonstrates the RNA expression of the ckit positive stem cell attachment to the calcified heart valve.

FIG. 9 depicts the results of testing the anti-inflammatory drug atorvastatin at 80 mg per day in a rabbit model of bioprosthetic valve calcification, with the control diet showing little atherosclerosis, the cholesterol diet demonstrating severe atherosclerosis and the Atorvastatin therapy with the cholesterol diet demonstrating attenuation of the atherosclerosis.

### DETAILED DESCRIPTION OF THE INVENTION

The invention provides a method for inhibiting stenosis, obstruction, or calcification of a native valve, a stented aorta and valve leaflet or bioprosthesis with or without a sewing ring, following implantation of a valve prosthesis in a patient in need thereof, which may include treatment with a oral medical therapy for valvular heart disease that has evidence of early to late evidence disease, as soon as the deployment of the elastical stent, gortex covering, and the bioprosthesis wherein the oral therapy with one or more therapeutic agents alone or in combination to improve the efficacy of the inhibition of calcification and the improvement of the longevity of the prosthetic material including the stent, the valve, and the gortex covering specifically to slow the progression of bicuspid aortic valve (BAV) calcification, Tricuspid aortic valve calcification (TAV), transcutaneous aortic valve replacement (TAVR), Surgical Bioprosthetic aortic valve replacement (SBAVR), mitral valve myxomatous degeneration (MVMD) via the activation of the Wnt pathway via the cleavage of Notch1 protein and the phosphorylation of glycogen synthase kinase which in turn releases beta catenin to the nucleus to activate bone and cartilage formation the heart valve and or prosthesis and this invention will include several therapeutic medical therapies to slow the progression of stenosis, obstruction, calcification and or regurgitation of the mitral valve.

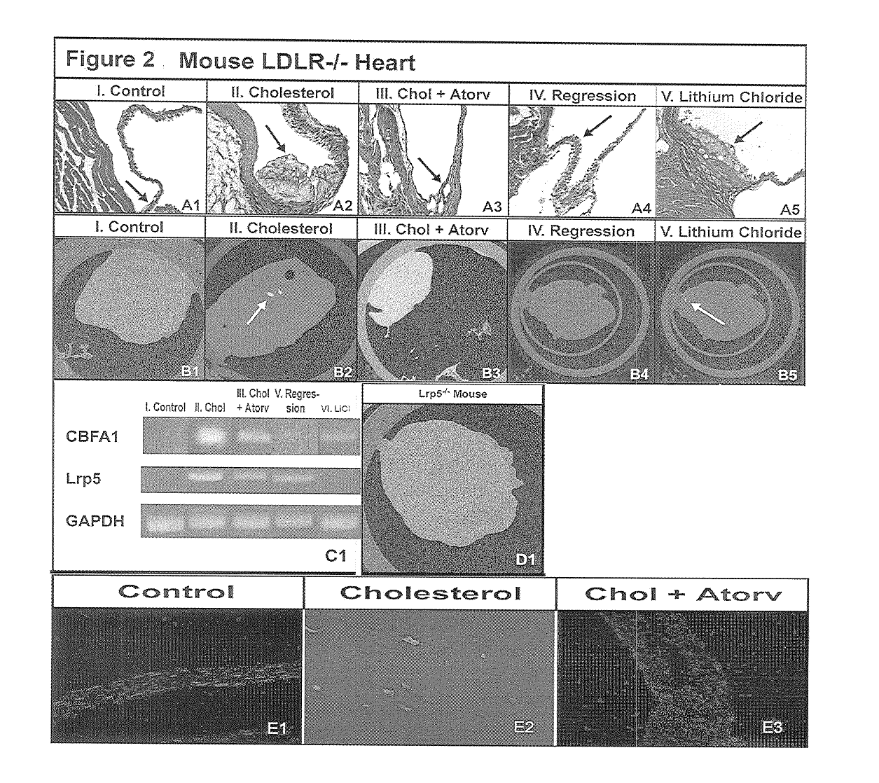

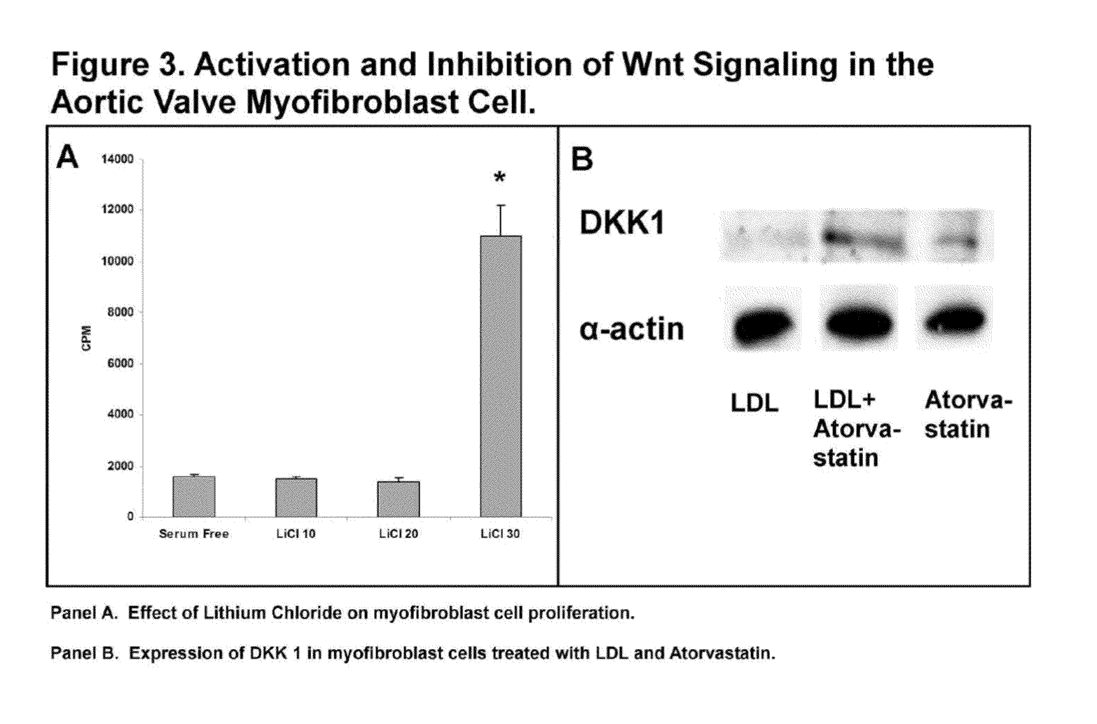

The inventor has also developed a method for inhibiting stenosis, obstruction, or calcification of a native heart valve and bioprosthetic valve following surgical implantation of said bioprosthetic valve in a vessel having a wall is disclosed herein. A patient is provided with a series of medical treatments alone or in combination as the native valve develops valvular disease and at the moment of bioprosthetic valve for surgical replacement of a natural diseased valve. The bioprosthetic valve may include an elastical stent via the activation of osteogenic bone and cartilage formation in the native valve leaflets and or the bioprosthetic valve leaflet after the attachment of a mesenchymal stem cell with the potential for osteogenic bone formation (as best seen in FIG. 2), the development of native valve atherosclerosis in the presence of a cholesterol diet, lithium chloride diet, and the attenuation of the valve leaflet with the treatment of atorvastatin in a mouse valve leaflet that has no LDL receptors. FIG. 3 demonstrates the effect of the direct treatment of Lithium Chloride on myofibroblast cells in the activation of cell proliferation and the inactivation of DKK1 in the presence of atorvastatin.

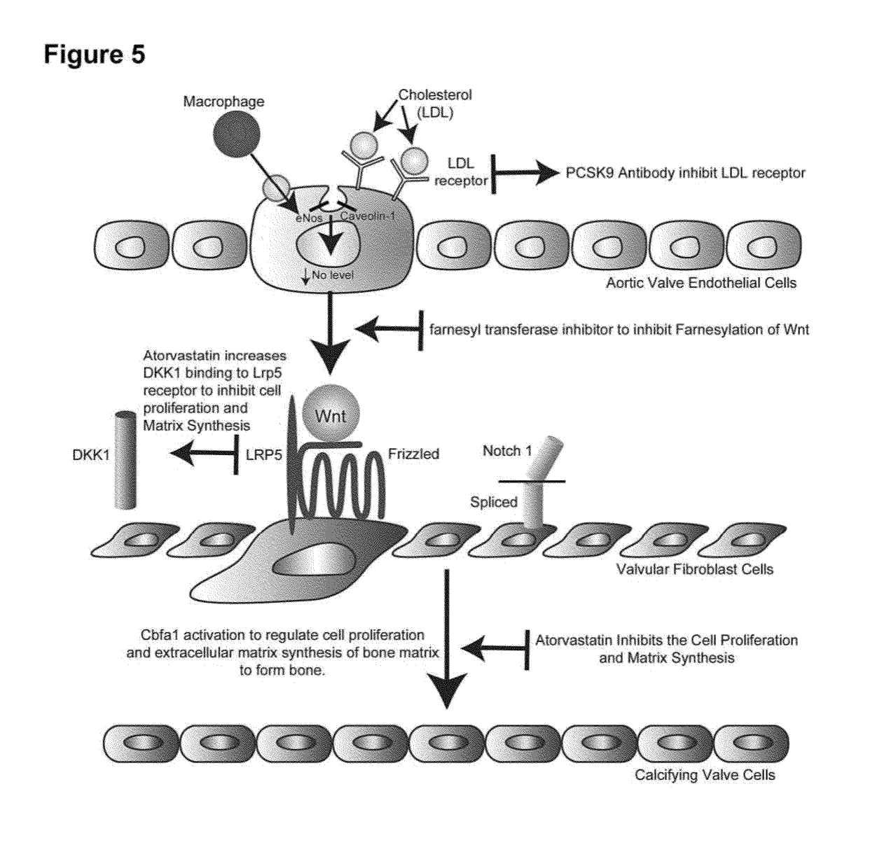

A method to inhibit the splicing of the Notch1 Receptor by treating the valve with lipid lowering agents statins in combination with PCSK9 antibody which will inhibit the LDL receptor to modulate the lipid levels is also provided herein. Farsnesyltransferase (“FTI”) inhibitors inhibit the farsnesylation of Wnt to inhibit the binding of Wnt3a to LRP5 receptor which modulates the myofibroblast to differentiate via the osteogenic bone pathway in the presence of hyperlipidemia. FTI inhibitors are small molecules which reversibly bind to the farnesyltransferase CAAX binding site. An FTI inhibitor will inhibit the activation of Wnt3a in cell attachment to form disease in the prosthetic valve leaflet and or native valve cell proliferation and or bone formation by decreasing farnesylation of Wnt3a which is critical for the activation of the Wnt3a/LRP5/Frizzled complex as demonstrated in FIG. 5

Therapeutic agents that inhibit cell proliferation and calcification in combination with an effective amount of a statin and a PCSK9 antibody inhibits cell attachment and or native valve cell proliferation and or bone formation by decreasing inflammation in state of hyperlipidemia. PCSK9 is a regulator of plasma lipoprotein cholesterol (LDL-C). The proprotein convertase subtilisin/kexin type 9 (PCSK9) protein regulates the activity of low-density lipoprotein (LDL) receptors. Inhibition of PCSK9 with a monoclonal antibody results in increased cycling of LDL receptors and increased clearance of LDL cholesterol (LDL-C). Highly expressed in the liver, PCSK9 is secreted after the autocatalytic cleavage of the prodomain, which remains non-covalently associated with the catalytic domain as indicated in FIG. 5, which inhibits the LDLR receptor via the PCSK9 antibody in combination with a statin agent. These therapeutic agents inhibit cell proliferation and calcification in combination with an effective amount of a farsnesyltransferase inhibitor (FTI) which inhibits the activation of Wnt3a in cell attachment to form disease in the prosthetic valve leaflet and or native valve cell proliferation and or bone formation by decreasing farnesylation of Wnt3a which is critical for the activation of the Wnt3a/LRP5/Frizzled complex as demonstrated in FIG. 5.

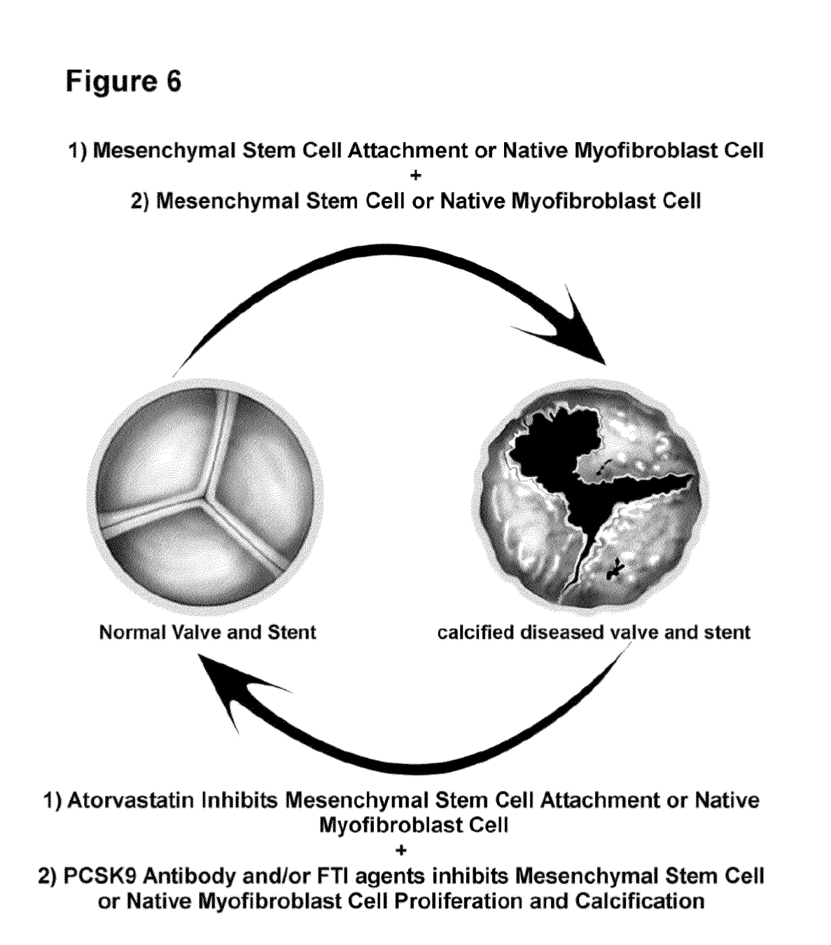

The treatments and methods provided herein in combination with the therapeutic agents disclosed herein in patients with native valve disease and or a bioprosthetic valve either placed surgically or transcutaneous will slow the progression of calcification, stenosis, regurgitation, and obstruction inhibit and/or slow the progression of stenosis, obstruction, and/or calcification of the bioprosthesis or the natural valve or both following implantation of the bioprosthetic valve as shown in FIG. 6.

Securing a bioprosthetic collapsible elastical valve which is mounted on the elastical stent at a desired position in the patient such that the elastical stent is in contact with a natural valve that may or may not have been surgically removed, and optionally treating with a medical therapy to inhibit the attachment of stem cells capable of developing calcification on both sides of the valve leaflets, the stent or a sewing ring to which the bioprosthetic valve is secured thereby inhibiting stenosis, obstruction, or calcification of the stented aorta following implantation of the stented valve prosthesis or in a patient in need thereof or the surgical replacement of a bioprosthesis that replaces a native valve or in patients who have early to late valvular disease process.

As used herein, the term “stenosis” may refer to the narrowing of a heart valve that could block or obstruct blood flow from the heart and cause a back-up of flow and pressure in the heart. Valve stenosis may result from various causes, including, but not limited to, scarring due to disease, such as rheumatic fever; progressive calcification via bone formation on the leaflet; progressive wear and tear; among others.

As used herein, the term “valve” may refer to any of the four main heart valves that prevent the backflow of blood during the rhythmic contractions. The four main heart valves are the tricuspid, pulmonary, mitral, and aortic valves. The tricuspid valve separates the right atrium and right ventricle, the pulmonary valve separates the right atrium and pulmonary artery, the mitral valve separates the left atrium and left ventricle, and the aortic valve separates the left ventricle and aorta.

In an embodiment of the method, the bioprosthetic valve and the diseased valve may be an aortic valve, pulmonary valve, tricuspid valve, or mitral valve.

As used herein, the term “valve prosthesis” may refer to a device used to replace or supplement a heart valve that is defective, malfunctioning, or missing. Examples of valve prostheses include, but are not limited to, bioprostheses; mechanical prostheses, and the like including, ATS 3fs® Aortic Bioprosthesis, Carpentier-Edwards PERIMOUNT Magna Ease Aortic Heart Valve, Carpentier-Edwards PERIMOUNT Magna Aortic Heart Valve, Carpentier-Edwards PERIMOUNT Magna Mitral Heart Valve, Carpentier-Edwards PERIMOUNT Aortic Heart Valve, Carpentier-Edwards PERIMOUNT Plus Mitral Heart Valve, Carpentier-Edwards PERIMOUNT Theon Aortic Heart Valve, Carpentier-Edwards PERIMOUNT Theon Mitral Replacement System, Carpentier-Edwards Aortic Porcine Bioprosthesis, Carpentier-Edwards Duraflex Low Pressure Porcine Mitral Bioprosthesis, Carpentier-Edwards Duraflex mitral bioprosthesis (porcine), Carpentier-Edwards Mitral Porcine Bioprosthesis, Carpentier-Edwards S.A.V. Aortic Porcine Bioprosthesis, Edwards Prima Plus Stentless Bioprosthesis, Edwards Sapien Transcatheter Heart Valve, Medtronic, Freestyle® Aortic Root Bioprosthesis, Hancock® II Stented Bioprosthesis, Hancock II Ultra® Bioprosthesis, Mosaic® Bioprosthesic, Mosaic Ultra® Bioprosthesis, St. Jude Medical, Biocor®, Biocor™ Supra, Biocor® Pericardia, Biocor™ Stentless, Epic™, Epic Supra™, Toronto Stentless Porcine Valve (SPV®), Toronto SPV II®, Trifecta, Sorin Group, Mitroflow Aortic Pericardial Valve®, Cryolife, Cryolife aortic Valve® Cryolife pulmonic Valve®, Cryolife-O'Brien stentless aortic xenograft Valve®

Generally, bioprostheses comprise a valve having one or more cusps and the valve is mounted on a frame or stent, both of which are typically elastical. As used herein, the term “elastical” means that the device is capable of flexing, collapsing, expanding, or a combination thereof. The cusps of the valve are generally made from tissue of mammals such as, without limitation, pigs (porcine), cows (bovine), horses, sheep, goats, monkeys, and humans.

According to the method of the present invention, the valve may be a collapsible elastical valve having one or more cusps and the collapsible elastical valve may be mounted on an elastical stent.

In an embodiment, the collapsible elastical valve may comprise one or more cusps of biological origin.

In another embodiment, the one or more cusps are porcine, bovine, or human.

Examples of bioprostheses may comprise a collapsible elastical valve having one or more cusps and the collapsible elastical valve is mounted on an elastical stent include, but are not limited to, the SAPIEN transcatheter heart valve manufactured Edwards Lifesciences, and the CoreValve® transcatheter heart valve manufactured by Medtronic and Portico-Melody by Medtronic.

The elastical stent portion of the valve prosthesis used in the present invention may be self-expandable or expandable by way of a balloon catheter. The elastical stent may comprise any biocompatible material known to those of ordinary skill in the art. Examples of biocompatible materials include, but are not limited to, ceramics; polymers; stainless steel; titanium; nickel-titanium alloy, such as Nitinol; tantalum; alloys containing cobalt, such as Elgiloy® and Phynox®; and the like.

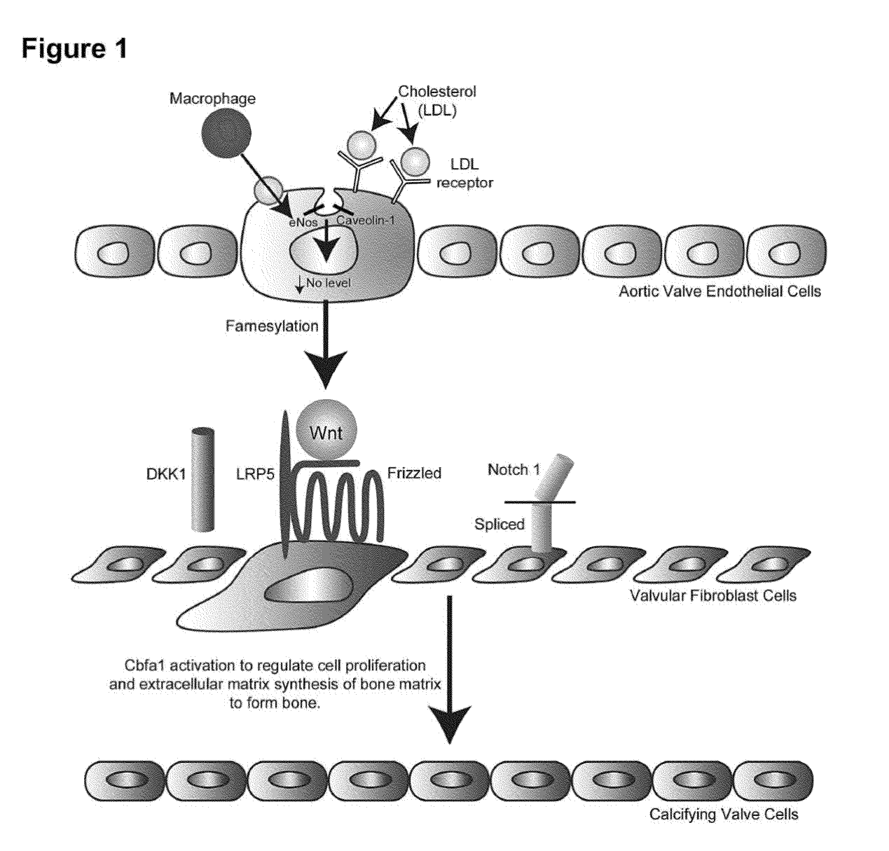

According to the method of the present invention, oral treatment of a patients with one or more therapeutic agents in combination to inhibit the development of valve calcification which develops in FIG. 1, in the presence of hyperlidemia, there is a decrease in Nitric oxide and Wnt3a is farnesylated in order for the secretion of Wnt, which in turns binds to Lrp5, in addition Notch1 is spliced and inactivated in order for the initiation of cell proliferation and the initiation of cell proliferation via activation of CBFA1 and the initiation of bone formation by activation of osteogenic bone program as listed in Table I.

Once the activation of the bone formation within the valve leaflet myofibroblast cell and or stem differentiation to bone formation as it attaches to valve prosthesis and or the elastical stent attached to a bioprosthesis. The elastical stent portion of the valve prosthesis may be any shape cylindrical (final shape is cylinder may be funnel shaped original all required to contact the valve or walls of the valve where, without being bound to theory, the therapeutic agents are released and absorbed by the valve or walls of the valve, or the aorta including aortic valve, mitral valve, tricuspid valve, vena cava valve.

In an embodiment, the elastical stent portion may be substantially cylindrical so as to be able to contact the valve or walls of the valve upon securing.

In another embodiment, the diameter of the elastical stent portion may be about 15 mm to about 42 mm.

According to an embodiment of the present invention, the method further may comprise introducing a nucleic acid encoding a nitric oxide synthase into the one or more cusps of the valve prosthesis. Methods for introducing a nucleic acid encoding a nitric oxide synthase into the one or more cusps are described in U.S. Pat. No. 6,660,260, issued Dec. 9, 2003, and is hereby incorporated by reference in its entirety.

As best seen in FIG. 1 several therapeutic medical therapies slow the progression of stenosis, obstruction, calcification and or regurgitation of the mitral valve. In the presence of hyperlipidemia, there is a decrease in Nitric oxide and Wnt3a is farnesylated in order for the secretion of Wnt, which in turns binds to Lrp5, in addition Notch1 is spliced and inactivated in order for the CBFA1 regulation of cell proliferation extracellular matrix protein synthesis to initiate bone formation by activation of osteogenic bone program.

As best seen in FIG. 2, an experimental hypercholesterolemic diet was given to mice which were genetically modified with the absence of the low-density-lipoprotein receptor, FIG. 2, Panel A1 is the control diet, FIG. 2, Panel A2 is the Cholesterol diet, FIG. 2, Panel A3 is the Cholesterol+Atorvastatin diet with improvement in the atherosclerosis, FIG. 2, Panel A4 is the regression diet with the treatment with cholesterol and then half way through the diet Atorvastatin treatment, and FIG. 2, Panel A5 the treatment with Lithium Chloride diet induced an atherosclerotic lesion in the absence of cholesterol, but with the inhibition of Glycogen synthase kinase to inhibit the Lrp5/beta catenin pathway. FIG. 2, Panel B1-B5 is the microCT data from the corresponding diets in the valve leaflets defined in FIG. 2, Panel A, Panel B1 control diet has no evidence of calcification, FIG. 2, Panel B2 the cholesterol diet demonstrates increase in calcification, FIG. 2, Panel B3 and B4 atorvastatin treatments has no evidence of calcification and FIG. 2, Panel B5 with the lithium Chloride diet demonstrates micro calcification in the heart valve. FIG. 2, Panel C1 demonstrates the gene expression of the increase in the bone transcription factor CBFA1 in the cholesterol treatment and Lrp5 gene expression. The Lrp5 null mouse has no evidence of calcifications in the heart. FIG. 2, Panel E is the confocal microscopy of the stain for beta catenin, which translocates to the nuclei to activate bone formation downstream of Lrp5. FIG. 2, Panel E1 demonstrates the positive translocation of beta-catenin to the nuclei in the treatment of cholesterol diet.

FIG. 3, Panel A depicts the in vitro data of the direct treatment of myofibroblast cells with the increasing dose of lithium chloride increasing cell proliferation.

FIG. 3, Panel B is the inhibition of DKK1 with atorvastatin and the direct inhibition of Lrp5.

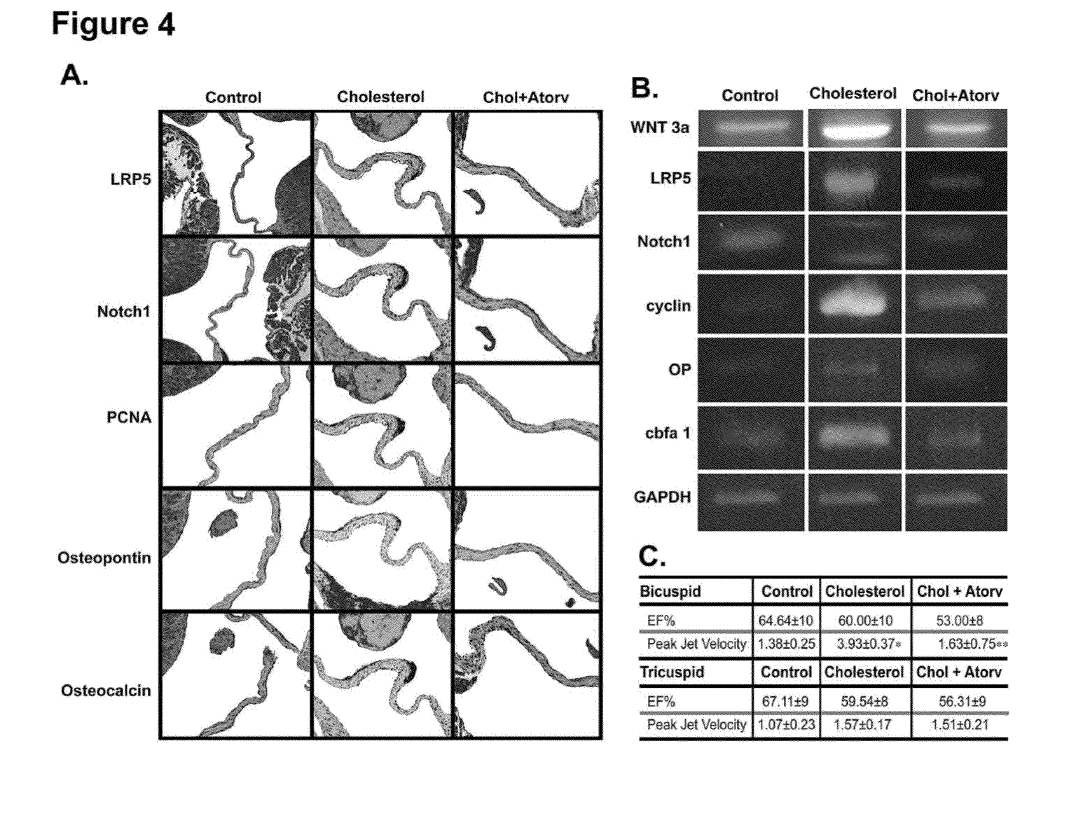

FIG. 4, demonstrates the characterization of the eNOS phenotype as defined by histology, RTPCR and echocardiography. In FIG. 4, Panel A is the histology for BAV, FIG. 4, Panel B is the semi-quantitative RTPCR from the BAV eNOS^{-/-} mice, and echocardiographic data for the bicuspid vs. tricuspid aortic valves. Referring to FIG. 4, Panel C, to understand if eNOS^{-/-} mice with the BAV phenotype, develops accelerated stenosis earlier than tricuspid aortic valves via the Lrp5 pathway activation, eNOS^{-/-} mice were given a cholesterol diet versus cholesterol and atorvastatin. Echocardiography hemodynamics was also performed to determine the timing of stenosis in bicuspid vs. tricuspid aortic valves eNOS^{-/-} mice on different diets. To further understand the mechanism of bicuspid aortic valve disease, eNOS null mouse was tested in a large number of mice (n=60) with control diet, (n=60) cholesterol diet and (n=60) cholesterol+Atorvastatin diet. Prior to sacrifice echocardiography for the presence and absence of a bicuspid aortic valve and FIG. 4, Panel C is the echocardiography from the eNOS null mouse on the different diets. We measured Notch1, Wnt3a and downstream markers of the canonical Wnt pathway by protein and RNA expression. Notch1 protein was diminished and the RNA expression demonstrates a similar spliced variant with lipid treatments, which was not present with the control and atorvastatin treatment. Cholesterol diets increased the members of the canonical Wnt pathway and Atorvastatin diminished these markers significantly (p<0.05).

As noted in FIGS. 1-5 the role of activation of the Lrp5 Wnt pathway in the development of this disease process, specifically in the genetic mouse lacking the LDL receptor is shown. The development of atherosclerosis in the presence of the Lithium Chloride diet which activates the beta catenin to initiate the cell proliferation and extra cellular matrix production is also shown.

Referring now to FIG. 5 a schematic view showing the cell layers which develop the disease process in the native valve leaflet are depicted. Signaling between the endothelial cell to the myofibroblast cell in the presence of hyperlipidemia activates the secretion of Wnt to turn on the Lrpb5 receptor, which in turn activates bone formation in the native myofibroblast cell. Different inhibitors and oral agents listed in Table I inhibit or slow the progression of disease.

FIG. 5 further depicts the role of PCSK9 as a regulator of plasma lipoprotein cholesterol (LDL-C) and as an agent that is effective in risk reduction in coronary artery disease. The proprotein convertase subtilisin/kexin type 9 (PCSK9) protein regulates the activity of low-density lipoprotein (LDL) receptors. Inhibition of PCSK9 with a monoclonal antibody results in increased cycling of LDL receptors and increased clearance of LDL cholesterol (LDL-C). Highly expressed in the liver, PCSK9 is secreted after the autocatalytic cleavage of the prodomain, which remains non-covalently associated with the catalytic domain. The catalytic domain of PCSK9 binds to the epidermal growth factor-like repeat A (EGF-A) domain of low density lipoprotein receptor (LDLR). Both functionalities of PCSK9 are required for targeting the LDLR-PCSK9 complex for lysosomal degradation and lowering LDL-C, which is in agreement with mutations in both domains linked to loss-of-function and gain-of-function¹.

The present invention provides for therapeutic regimens for prolonged reduction of LDL-C levels in blood by inhibiting PCSK9 activity and the corresponding effects of PCSK9 in combination with a statin agent as outlined in Table I below with a statin agent on LDL-C plasma levels in patients who have aortic valve disease, mitral valve prolapse and or bioprosthetic valves, including transcutaneous aortic valve replacements.

Table I demonstrates the different oral therapies single and in combination to treat the slow the progression of bicuspid aortic valve (BAV) calcification, Tricuspid aortic valve calcification (TAV), transcutaneous aortic valve replacement (TAVR), Surgical Bioprosthetic aortic valve replacement (SBAVR), mitral valve myxomatous degeneration (MVMD) via the activation of the Wnt pathway via the cleavage of Notch1 protein and the phosphorylation of glycogen synthase kinase which in turn releases beta catenin to the nucleus to activate bone and cartilage formation the heart valve and or prosthesis and this invention will include several therapeutic medical therapies to slow the progression of stenosis, obstruction, calcification and or regurgitation of the mitral valve. Anti-hyperlidemic agents including combination with an effective amount of Atorvastatin in the range of 10 mg to 80 mg, Simvastatin in the range of 10 mg to 40 mg, Rosuvastatin 5 mg to 40 mg, Pravastatin 20 mg to 80 mg, Pitavastatin 1 mg to 4 mg and a PCSK9 antibody the initial dose can be about 0.25 mg/kg, about 0.5 mg/kg, about 1 mg/kg or about 1.5 mg/kg, and the initial dose and the first subsequent dose and additional subsequent doses can be separated from each other in time by about one week and or in combination with an FTI inhibitor such as Lonafarnib at a 115 mg/m2 dose with a range from 115 mg/m² to 150 mg/m², in combination with an effective amount of Zetia of 10 mg. Other effective FTI inhibitors include Chaetomellic acid A, FPT Inhibitor I, FPT Inhibitor II, FPT Inhibitor III, FTase Inhibitor I, FTase Inhibitor II, FTI-276 trifluoroacetate salt, FTI-277 trifluoroacetate salt, GGTI-297, Gingerol, Gliotoxin, L-744,832 Dihydrochloride, Manumycin A, Tipifarnib, a-hydroxy Farnesyl Phosphonic Acid.

TABLE 1

| | | | | | | Reduces blood |
|---|---|---|---|---|---|---|
| | | | | | | cholesterol by |
| | | | | | | inhibiting the |
| | | | LRP5 | | | absorption of |
| | LDL | Fameseylation | Receptor | Cell | Extracellar | cholesterol by the |
| Treatment | Receptor | Wnt | via DKK1 | Proliferation | Matrix Protein | small intestine |
| Statin Agents: | | | | | | |
| Atorvastatin in the range of 10 mg to 80 mg | XX | | XX | XX | XX | |
| Simvastatin in the range of 10 mg to 40 mg | | | | | | |
| Rosuvastatin 5 mg to 40 mg | | | | | | |
| Pravastatin 20 mg to 80 mg | | | | | | |
| Pitavastatin 1 mg to 4 mg | | | | | | |
| FTI Inhibitor | | | | | | |
| FTI | | XX | | | | |
| Ionafamib 115 mg/m² dose with a | | | | | | |
| range from 115 mg/m² to 150 mg/m² | | | | | | |
| PCSK9 Antibody | | | | | | |
| The initial dose can be in a range from | XX | | | | | |
| 0.25 mg/kg to 1.5 mg/kg, and the | | | | | | |
| initial dose and the first subsequent dose | | | | | | |
| and additional subsequent doses | | | | | | |
| can be separated from each other in | | | | | | |
| time by about one week | | | | | | |
| Zetia 10 mg a day | | | | | | XX |
| Statins and/or: | | | | | | |
| FTI | XX | XX | XX | XX | XX | XX |
| PCSK9 Antibody | | | | | | |
| Zetia | | | | | | |

FIG. 2 demonstrates the data to define the role of cholesterol in the activation of Lrp5 receptor and valve calcification experiments demonstrate atherosclerosis and calcification is developing in the aortic valves of the LDLR^{-/-} mice. This data characterizes the hearts in these mice to determine if the lipids affected the bone formation in these tissues and if statins can improve the bone biology. Results from this study arose from the following five treatment groups: Group I: the control diet, Group II: experimental hypercholesterolemia 0.2% (v/v) diet and Group III: experimental hypercholesterolemia 0.2% (v/v) diet, and Atorvastatin 0.1% (v/v) for 12 weeks, Group IV: 6 week cholesterol diet and then 6 weeks of Atorvastatin 0.1% (v/v) alone as a regression therapy group, and Group V: Lithium Chloride Diet 0.12% (v/v) alone, (N=20 per treatment group). Masson Trichrome stains were performed on the aortic valves to assess for atherosclerosis and early mineralization.

FIG. 2, is a composite of the Masson Trichrome light microscopy (40×) and MicroCT data from the aortic valves from the 5 different treatment groups. FIG. 2, Panel A1, shows that the control aortic valve does not develop any evidence of atherosclerosis. FIG. 2, Panel A2, demonstrates that the hypercholesterolemic aortic valve develops an atherosclerotic lesion which is calcified. The lesion develops along the aortic surface of the aortic valve. FIG. 2, Panel A3, is the aortic valve from the cholesterol plus atorvastatin treatment group which shows a marked improvement in the atherosclerotic lesion along the valve leaflet. FIG. 2, Panel A4, shows that the Group IV regression treatment aortic valves do not have any evidence of atherosclerosis along the aortic valve surface. FIG. 2, Panel A5 demonstrates the effects of Lithium Chloride a direct inhibitor of Glycogen Synthase Kinase. Treatment with Lithium Chloride increases the beta catenin levels within the cells and therefore turns on bone formation via translocation of beta catenin to the nucleus and activation of the LEF/TCF transcription factors. This data demonstrates evidence of an atherosclerotic lesion in the lithium chloride aortic valves. Arrow in A5 points to the atherosclerotic lesion. The ex vivo microCT analyses to show if calcification develops in the aortic valves from the different treatment groups. The LDLR^{-/-} mice were treated with cholesterol with and without atorvastatin as outlined above and scanned ex vivo after sacrifice in the MicroCT scanner. FIG. 2, Panel B, shows the preliminary microCT data from the LDLR^{-/-} mice from the five treatment groups. The areas of white in each figure indicate evidence of calcification in the valve leaflet. The grey area is the uncalcified area in each valve leaflet. The blue area is the background from the computer rendering for this data. FIG. 2, Panel B1, is the control diet (Group I) in which the aortic valves did not develop any evidence of calcification. The cholesterol (Group II) treated mice develop areas of early mineralization as shown by the two white areas of calcification present in the MicroCT scan shown in FIG. 2, Panel B2. The atorvastatin (Group III) treated hearts did not develop any calcification as shown in FIG. 2, Panel B3. FIG. 2, Panel B4, shows that the regression treatment (Group IV) aortic valves which also did not develop any evidence of mineralization. FIG. 2, Panel C1, demonstrates the RTPCR data for the different treatment groups. The RTPCR shows an increase in cbfa1 and Lrp5 receptor gene expression with the cholesterol diet (Group II), and atorvastatin treatment decreased the Cbfa1 and Lrp5 expression in the aortic valves in both the 12 week treatment with Atorvastatin, (Group III), and further decreased the Cbfa1 and Lrp5 gene expression in the 6 week Atorvastatin Regression treatment, (Group IV). Finally, the lithium chloride treatment demonstrated an increase in the Cbfa1 without any Lrp5 expression. The control diet (Group I) showed no Lrp5 expression and no cbfa1 expression. FIG. 2, Panel D1, is the control Lrp5^{-/-} treated mice. There was no evidence of calcification in the Lrp5^{-/-} mice^{2,3}.

Finally, confocal microscopy to examine beta-catenin expression in the aortic valves. FIG. 2, Panel E demonstrates the confocal microscopy of beta-catenin expression in three of the diet groups. Panel E1, shows little cytoplasmic beta-catenin expression in the control valves. Panel E2, shows the increase in the beta-catenin expression located in the nucleus and Panel E3, demonstrates attenuation of the beta-catenin protein expression.

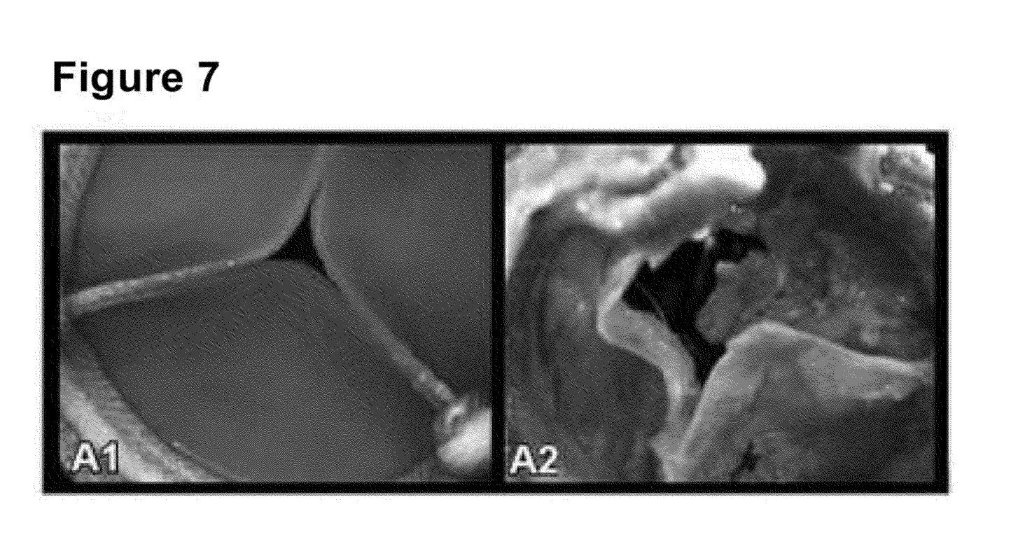

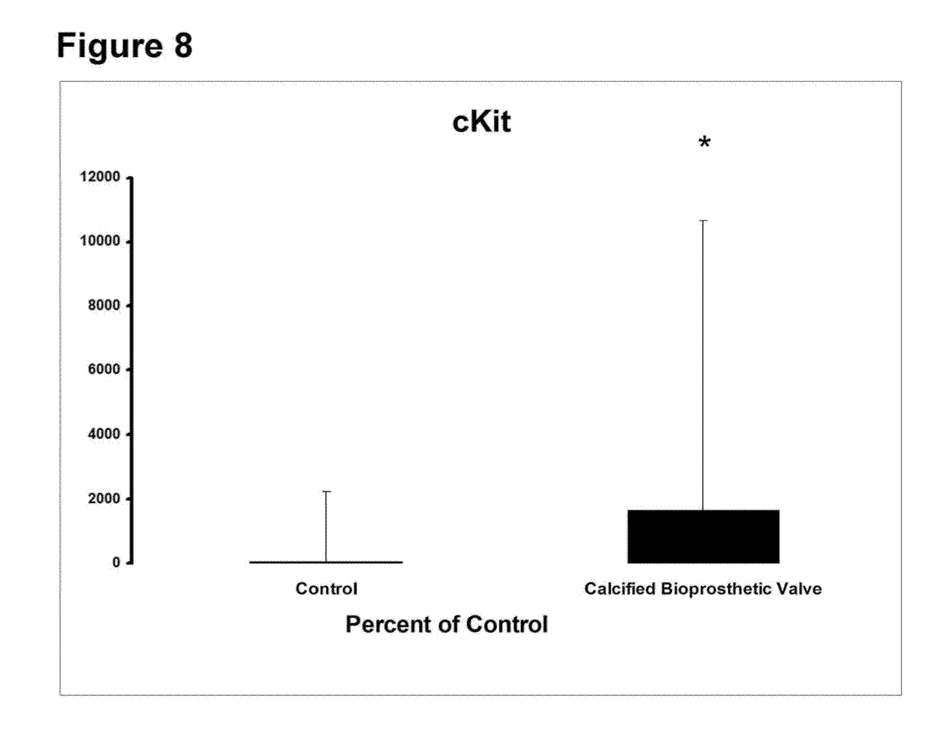

FIG. 6-9 depicts the pannus formation and calcification process in the explanted valves from human patients at the time of surgical valve replacement of a failed bioprosthetic heart valve. Control bioprosthetic valve versus explanted bioprosthetic valve from humans. Panel (a1) Ventricular surface of the control valve, (a2) ventricular surface of the diseased valve with the pannus and calcification process via a stem call attachment to the heart valve. FIG. 7 is a graph which demonstrates the RNA expression of the ckit positive mesenchymal stem cell attachment to the calcified heart valve, causing the calcification process to occur on the valve as expressed by the well known bone transcription factor cbfa1 (core binding factor a1) and opn (osteopontin) and extracellular matrix protein. The results are expressed as a percent of control with the control being 0 for all of these markers. GAPDH is a house keeping gene used as a control for the experiment. FIG. 8 demonstrates the increase in the cKit gene expression in the diseased bioprosthetic valve as compared to the control.

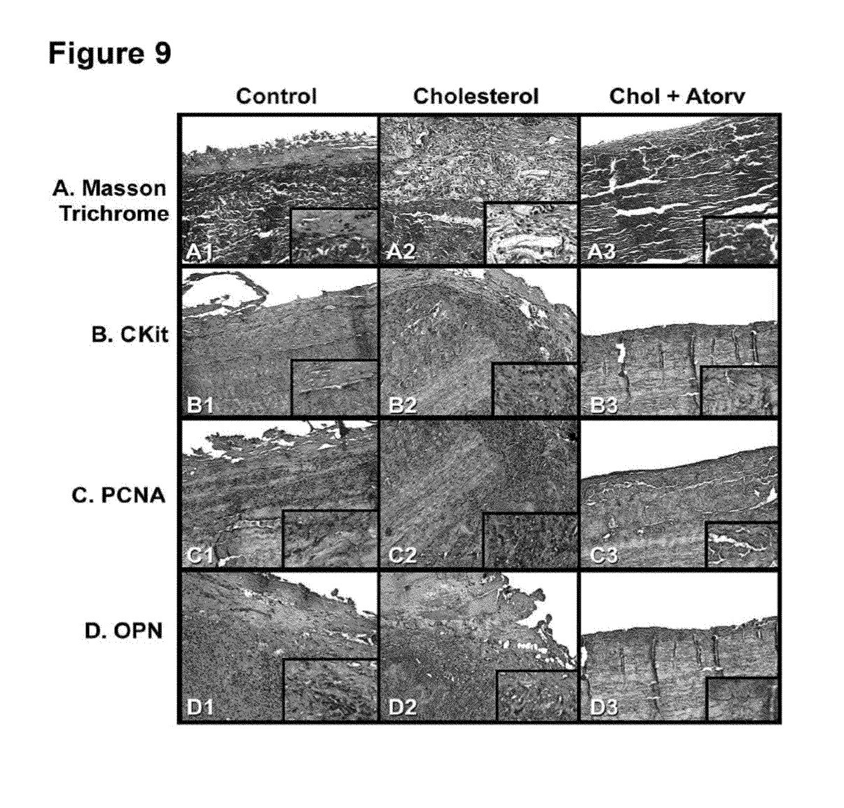

FIG. 9 The implanted valve leaflets from the control animals appeared to have a mild amount of cellular infiltration along the surface of the leaflet as demonstrated by Masson Trichrome stain FIG. 9, Panel A1. The high power magnification demonstrates the demarcation between the leaflet and cellular infiltrate that develops along the leaflet surface. There was a small amount of cKit positive staining cells in control bioprosthetic valves FIG. 9, Panel B 1, as well as a mild amount of proliferating cells expressing osteopontin, as shown in FIG. 9, Panels C1 and D1. In contrast, in the valve tissue from the cholesterol-fed rabbits FIG. 9, Panels A2, B2, C2, D2, there was marked cellular infiltrate as shown in the Masson Trichrome, the tissue infiltrate express cKit, PCNA and OPN. Finally, as measured by semi-quantitative visual analysis, at the time of explant and under the light microscopy, the atherosclerotic burden increased four-fold with the cholesterol treatment. The implanted leaflets in the atorvastatin treated rabbits demonstrated a marked decrease in the amount of atherosclerotic plaque burden, proliferation, cKit and osteopontin expression FIG. 9, Panels A3, B3, C3, D3.

FIG. 9 depicts results from an experimental animal to test for the dosing of the atorvastatin to reduce the inflammation and also the pannus formation on the valve leaflet. The experimental procedure is as follows, Male New Zealand white rabbits weighing 2.5-3.0 kg were assigned to a control (N=10) or 0.5% cholesterol-fed group (N=10) or to a cholesterol-fed and atorvastatin group (N=10). All animals were fed ad libitum for 12 weeks. Control rabbits were fed a standard diet. Cholesterol-fed animals received a diet supplemented with 0.5% (w/w) cholesterol (Purina Mills, Woodmont, Ind.), and the cholesterol-fed and atorvastatin group were given atorvastatin 3.0 mg/kg daily orally for the statin treatment arm. Prior to the initiation of the diet the rabbits underwent surgical implantation of bovine pericardial bioprosthetic valve tissue (Perimount, Edwards, Irvine Calif.) using intramuscular ketamine/xylazine (40/5 mg/kg). Following this 12-week period, the rabbits were anesthetized using intramuscular ketamine/xylazine (40/5 mg/kg) and then underwent euthanasia with intracardiac administration of 1 ml of Beuthanasia. Immediately after removal from the subcutaneous implantation site the bioprosthetic valves were fixed in 4% buffered formalin for 24 hours and then embedded in paraffin. Paraffin embedded sections (6 μm) were cut and stained with Masson Trichrome stain for histopathologic exam.

Table 2 below depicts the results of testing the anti-inflammatory drug atorvastatin at 80 mg per day equivalent to human dosing and shows the percent reduction of stem cell RNA expression on the valves treated with Atorvastatin and the reduction of stem cell mediated pannus formation.

TABLE 2

| | Control | Cholesterol | Chol + Atorv |
|---|---|---|---|
| Cholesterol | 18 ± 7 | 1846.3 ± 525.3* | 842 ± 152.1** |
| (mg/dl) | | | |
| Triglycerides | 102.3 ± 16.9 | 323.25 ± 274.6* | 97 ± 27.1** |
| (mg/dl) | | | |
| HDL (mg/dl) | 4.6 ± 5.1 | 21.3 ± 7.5* | 24.7 ± 16.7 |
| hsCRP (mg/dl) | 0.24 ± 0.1 | 13.6 ± 19.7* | 7.82 ± 8.7** |
| cKit | 681 ± 618 | 978 ± 1217 | 302 ± 290** |
| OPN | 1373 ± 1216 | 1662 ± 1491 | 1483 ± 1501 |
| Cyclin | 1461 ± 1339 | 2988 ± 3451* | 984 ± 970** |
| Sox9 | 580 ± 459 | 910 ± 554* | 168 ± 102** |
| GAPDH | 2233 ± 2887 | 2553 ± 3013 | 2456 ± 3158 |
| Morphometric | ++ | +++++ | +** |
| Measurement of | | | |
| Atherosclerosis | | | |
| Burden | | | |

Table 2 demonstrates the RNA gene expression for the control, cholesterol and cholesterol plus atorvastatin experimental assays. There was an increase in the Sox9, osteoblast transcription factor, Cyclin, and cKit in the leaflets of the cholesterol-fed animals as compared to the control and atorvastatin groups (p<0.05). Table I, is the RTPCR data from the experimental model. The serum cholesterol levels were significantly higher in the cholesterol fed compared to control assays (1846.0±525.3 mg/dL vs. 18.0±7 mg/dL, p<0.05). Atorvastatin treated experimental arm manifested lower cholesterol levels than the cholesterol diet alone (824.0±152.1 mg/dl, p<0.05). There was an increase in hsCRP serum levels in the cholesterol fed compared to control assays (13.6±19.7 vs. 0.24±0.1, p<0.05), which was reduced by atorvastatin (7.8±8.7, p<0.05). These assays were tested in a rabbit model of experimental hypercholestolemia with and without atorvastatin at a dose equivalent to 80 mg a day for humans. Previous experiments were performed to test the lower dosing ranges at 20 mg a day and 40 mg a day of Atorvastatin and there was zero therapeutic benefit at the lower dosing ranges.

Mechanisms of action for the role of statin as anti-inflammatory agent and antiprolifearative and anticalcific agent in combination will mediate the inhibition of calcification and stem cell attachment. Atorvastatin reduces the ckit stem cell from adhering to the valve to reduce further destruction of the valve by activating endothelial nitric oxide synthase in the valves in combination with the anti-proliferative agents. There was a 95% reduction in the myofibroblast proliferation and extracellular matrix production in the two models inhibiting calcification in these tissues, the use of a combination of drugs listed in Table I can inhibit various levels of activation of disease as outlined in FIGS. 1-9.

In addition, treatment may include using the aforementioned anti-hyperlidemic agents and PCSK9 antibody in combination with antiplatelet therapy such as aspirin and/or a P2Y12 inhibitor including Clopidogrel, Prasugrel, Ticagrelor. In the field of purinergic signaling, the P2Y12 protein is found mainly but not exclusively on the surface of blood platelets, and is an important regulator in blood clotting. P2Y12 belongs to the Gi class of a group of G protein-coupled (GPCR) purinergic receptors and is a chemoreceptor for adenosine diphosphate (ADP).

Doses that are effective to use in combination with treatment to prevent native and/or bioprosthetic heart valve calcification are Clopidogrel in a loading dose of 300 mg at the time of implantation and a maintenance dose of 75 mg/day thereafter; Prasugrel in a loading dose of 60 mg at the time of implantation and a maintenance dose of 10 mg/day thereafter; and Ticagrelor in a loading dose of 180 mg at the time of implantation and a maintenance dose of 90 mg two times per day thereafter.

Treatment of patients in accordance with the invention further inhibits the low density lipoprotein receptor in the endothelial cells in one or more cusps; the LRP5 receptor in the myofibroblast cells in one or more cusps and or mesenchymal stem cells and WNT3a secretion in endothelial cells in one or more cusps.

Although the present invention has been described with reference to various aspects and embodiments, those of ordinary skill in the art will recognize that changes may be made in form and detail without departing from the spirit and scope of the invention.

What is claimed is:

1. A method for inhibiting stenosis, obstruction, or calcification of a bioprosthetic valve implanted in a patient comprising:
implanting a bioprosthetic valve in a patient to replace a natural heart valve;
following implantation administering an effective amount of an anti-hyperlidemic agent in combination with a PCSK9 antibody; and
causing the inhibition of stenosis, obstruction, or calcification of the bioprosthetic valve or natural valve or both.

2. The method according to claim 1, wherein said effective amount of anti-hyperlidemic agents is selected from 10 mg to 80 mg of Atorvastatin, 10 mg to 40 mg of Simvastatin, 5 mg to 40 mg of Rosuvastatin, 20 mg to 80 mg of Pravastatin, 1 mg to 4 mg of Pitavastatin and combinations of the foregoing.

3. The method of claim 3 wherein an initial dose of PCSK9 is from 0.25 mg/kg to about 0.5 mg/kg.

4. The method of claim 3 wherein a subsequent dose of PCSK9 is from about 1 mg/kg to about 1.5 mg/kg.

5. The method of claim 4 wherein said initial dose and subsequent dose are separated in time by about one week.

6. The method of claim 1 further comprising administering an effective amount of a farnesyltransferase inhibitor.

7. The method according to claim 6 wherein said farnesyltransferase inhibitor comprises lonafarnib and said effective amount comprises from 115 mg/m² to 150 mg/m².

8. The method of claim 7 further comprising administering Zetia in an amount equal to of 10 mg.

9. The method of claim 1, wherein the bioprosthetic valve is an aortic bioprosthetic valve.

10. The method of claim 1, wherein the bioprosthetic valve is a bioprosthetic mitral valve.

11. The method of claim 1, wherein the bioprosthetic valve is a bioprosthetic pulmonic valve.

12. The method of claim 1, wherein the bioprosthetic valve is bioprosthetic tricuspid valve.

13. The method of claim 1, wherein the bioprosthetic valve comprises one or more cusps of biological origin.

14. The method of claim 13, wherein the one or more cusps is porcine, bovine, or human.

15. The method of claim 13, further comprising introducing a nucleic acid encoating a nitric oxide synthase into the one or more cusps.

16. The method according the claim 13, further comprising introducing a drug eluting treating encoating the one or more cusps on both sides with an anti-proliferative and anti-calcification treatment.

17. The method of claim 1 further comprising administering aspirin in an amount equal to 80 mg/day.

18. The method of claim 1 further comprising administering an effective amount of an oral P2Y12 inhibitor.

19. The method of claim 18 wherein said P2Y12 inhibitor is selected from Clopidogrel, Prasugrel, Ticagrelor and combinations of the foregoing.

20. The method of claim 19 wherein said effective amount of Clopidogrel is a loading dose of 300 mg at the time of implantation and a maintenance dose of 75 mg/day thereafter.

21. The method of claim 19 wherein said effective amount of Prasugrel is a loading dose of 60 mg at the time of implantation and a maintenance dose of 10 mg/day thereafter.

22. The method of claim 19 wherein said effective amount of Ticagrelor is a loading dose of 180 mg at the time of implantation and a maintenance dose of 90 mg two times per day thereafter.
